(19) United States
(12) Patent Application Publication
(10) Pub. No.: US 2014/0163498 A1
(43) Pub. Date: Jun. 12, 2014

(54) Urinary Device

(71) Applicant: Adam Christian Natusch, Edgartown, MA (US)

(72) Inventor: Adam Christian Natusch, Edgartown, MA (US)

(21) Appl. No.: 13/712,809

(22) Filed: Dec. 12, 2012

(51) Int. Cl. A61F 5/44

(52) U.S. Cl. CPC A61F 5/44
USPC 604/349

(57) ABSTRACT

A male urinary device comprises a thin, light weight, stretchable, and elongated tube that has a diameter of 2-3 cm and is open at both ends. The tube would be rolled onto a wearer's urinary organ and then extended down out the bottom of the wearer's pant leg so that the device allows a male to urinate without removing their clothes. The length of the tube varies to accommodate individuals' height. Preferably, the tube is between 90 cm and 100 cm. The urinary device may be made from stretchable and resilient materials including but not limited to latex so that it is comfortable to wear.

### REFERENCE TO RELATED APPLICATIONS

This patent application claims the benefit of U.S. Provisional Application No. 61/630,409 filed on Dec. 12, 2011, the disclosure of which is incorporated herein by reference in its entirety.

### BACKGROUND OF THE INVENTION

1. Field of the Invention

The present invention relates to a urinary device that is worn on the body and, more particularly, to a urinary device to be worn on the male urinary organ.

2. Description of Related Art

Often times people run into the problem of needing to use the bathroom but they aren't available. This is something that men who drink beer and go to sporting events often encounter and end up getting arrested for urinating in the public. People like taxi drivers who cannot stop their car in the city to use the bathroom also run into this problem. This is also a problem for men with weak bladders. Currently, adult diaper is a solution for men with weak bladders but there should be an alternative to it.

There are no urinary devices that are designed for healthy and normal men to wear for the situation mentioned earlier. Therefore, there exists a need for such a device.

### SUMMARY OF THE INVENTION

It is a primary object of the present invention to provide a device that enables males to urinate outdoors without the removal of clothing.

In an exemplary embodiment of the present invention, there is disclosed a urinary device, which comprises a tube with openings on both ends. The tube would be rolled onto a wearer's penis and then extended down and out of the bottom of the wearer's pant leg so that the device allows a male to urinate without removing his clothing.

The urinary device may be constructed of various types of stretchable and resilient materials, preferably, latex or other similar materials so that it will be comfortable on the penis and will stay secured on the penis.

In one embodiment, the urinary device may be beaded on both ends. In a second embodiment, the urinary device may be beaded on the penis insertion end and tapered on the urine exit end. In a third embodiment, the urinary device may be tapered slightly on the penis insertion end to enable the device to stay better secured on the penis and a greater tapered urine exit end. The greater tapering on the urine exit end ensures the flow of urine out of the device to be better controlled.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a perspective isomeric view of an embodiment of the urinary device.

FIG. 2 is a perspective view of an embodiment of package which contains a rolled-up urinary device therein.

FIG. 3 is a side view along the line of A-A of FIG. 1.

FIG. 4 is a perspective view of an embodiment of the urinary device with the penis insertion end slightly tapered and the urine exit end with greater tapering.

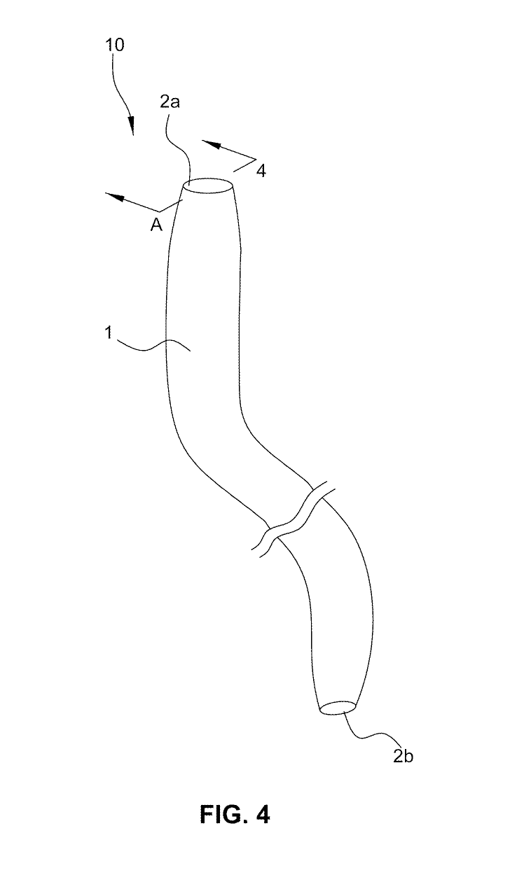

FIG. 5 is a side view along the line of A-A of FIG. 4.

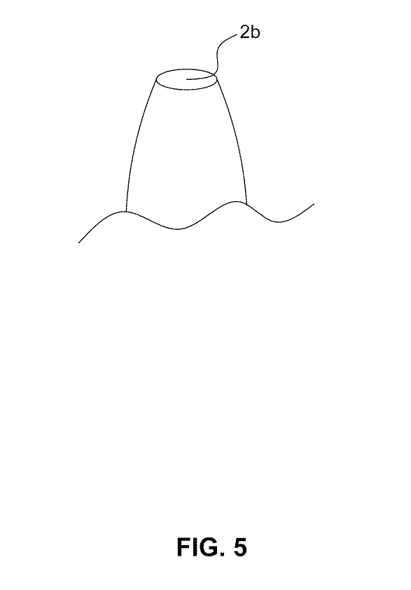

### DESCRIPTION OF THE PREFERRED EMBODIMENT

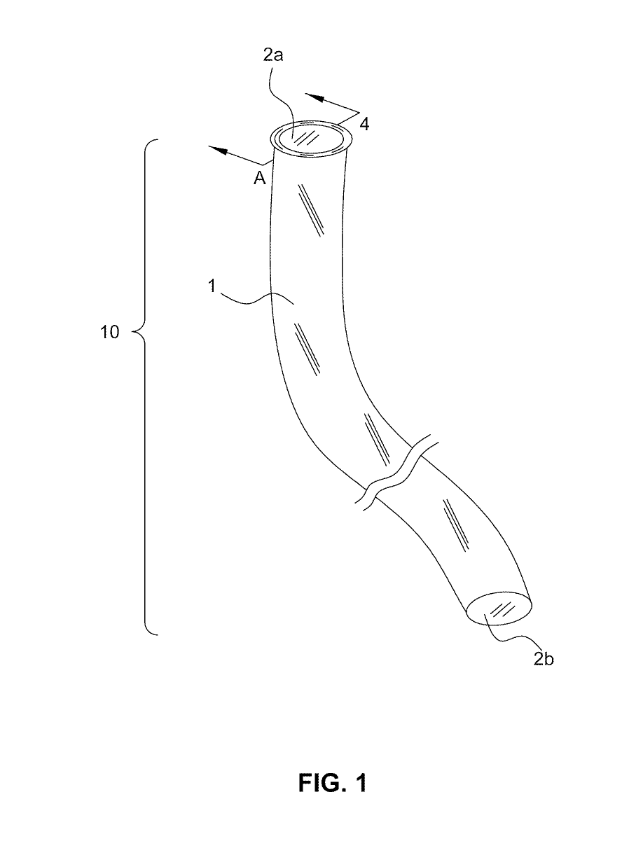

Referring to FIG. 1 there is disclosed a perspective view of an embodiment of the urinary device, denoted by the reference numeral 10, according to the present invention. The urinary device 10 comprises: a thin, lightweight elongated tube 1 with openings 2a and 2b at both ends.

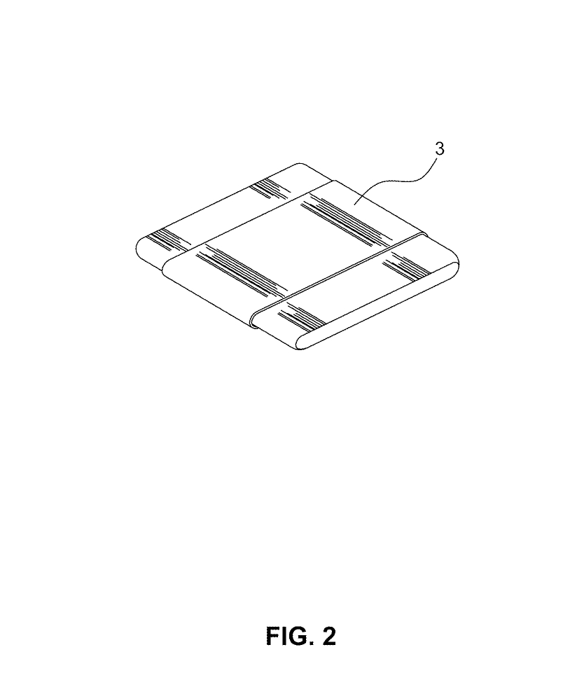

Referring to FIG. 2 there is disclosed a packaging 3 similar to the typical package of condoms. The package 3 containing a roll-up urinary device 10 therein is small, flat, light weight and easy to carry.

The urinary device 10 may be constructed of various types of material that is flexible, stretchable, resilient and thus is comfortable to wear. Preferably, the urinary device is made from latex which is the most popular materials for condom so that it can stay secured on the penis like a latex condom. The urinary device may also be made from non-latex material such as polyurethane or other synthetic materials.

The urinary device 10 is approximately 2-3 cm, preferably 2.5-3 cm, in diameter at both openings. This dimension is smaller than the openings of a condom allowing for the device to stay on the penis even when the penis is flaccid.

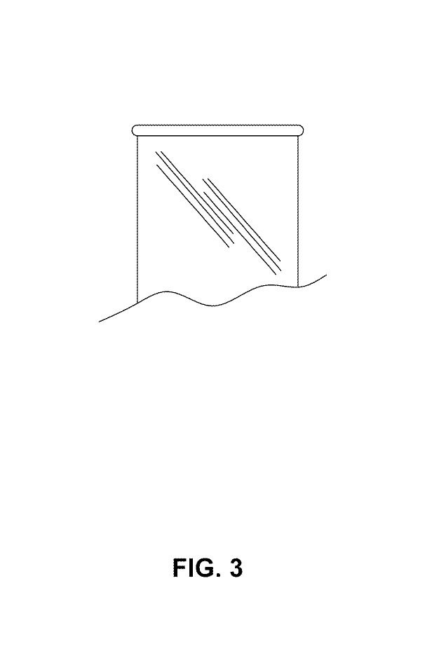

In another embodiment, the urinary device 10 may be beaded 4 on both ends 2a and 2b as shown in FIGS. 1 and 3.

In yet another embodiment, the urinary device 10 may utilize medical-grade adhesive (of the type commonly used for adhesive bandages) in the interior of the tube 1 to further help secure the device.

The length of the the urinary device 10 varies in order to accommodate different height of individuals. Typically, the urinary device 10 is approximately 80-110 cm in length. Preferably, the urinary device 10 is 90-100 cm long.

In a second embodiment, the urinary device 10 may be beaded 4 on the penis insertion end 2a and tapered on the urine exit end 2b.

In a third embodiment, the urinary device 10 may be tapered slightly on the penis insertion end 2a to enable the device to stay better secured on the penis and a greater tapered urine exit end 2b. The greater tapering on the urine exit end 2b ensures the flow of urine out of the device to be better controlled.

Furthermore, because the materials used for the urinary device is stretchable, the urinary device is adaptable and fits to the shape of the penis so it doesn't cause constriction, discomfort, or displacement of the device out of position, resulting in the device to work improperly. Moreover, due to the low cost of the materials used to make the urinary device of the present invention the production cost of the device is low enough to allow a user to replace the devices frequently like a condom.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

1. A urinary device comprising: a thin and lightweight elongated tube having a first end and a second end, both of the first end and second end are open; wherein the tube is rolled onto a wearer's penis from the first end and the rest of the tube extends downward with the second end extending out of a bottom of the wearer's pant leg to let the urine flow out.

2. The urinary device of claim 1, wherein the tube is 80-110 cm in length and 2-3 cm in diameter at both ends.

3. The urinary device of claim 1, wherein the tube is preferably 90-100 cm in length and 2.5-3 cm in diameter at both ends.

4. The urinary device of claim 1, wherein the tube is made from stretchable and resilient materials.

5. The urinary device of claim 4, wherein the tube is made from latex or polyurethane.

6. The urinary device of claim 1 further comprising medical-grade adhesive in the interior of the tube to further secure the tube.

7. The urinary device of claim 1, wherein the first end of tube is beaded.

8. The urinary device of claim 7, wherein the second end of the tube is beaded.

9. The urinary device of claim 1, wherein the first end of the tube is tapered to enable the device to stay better secured on the penis.

10. The urinary device of claim 1, wherein the second end of the tube is tapered to ensure the urine flowing out of the device to be better controlled.

11. A urinary device comprising:
a thin and lightweight elongated tube having a first end and a second end, both of the first end and second end are open;
wherein the tube is rolled onto a wearer's penis from the first end and the rest of the tube extends downward with the second end extending out of a bottom of the wearer's pant leg to let the urine flow out;
wherein said tube is 80-110 cm in length and 2-3 cm in diameter at both ends and
preferably 90-100 cm in length and 2.5-3 cm in diameter at both ends, made from stretchable and resilient materials, including but not limited to latex or polyurethane;
wherein the first end of tube is beaded and tapered to enable the device to stay better secured on the penis and the second end of the tube is beaded and tapered to ensure the urine flowing out of the device to be better controlled.
